(19) United States
(12) Patent Application Publication
(10) Pub. No.: US 2005/0049549 A1
(43) Pub. Date: Mar. 3, 2005

(54) Method and device for enhancing transdermal agent flux

(76) Inventors: Patrick S.L. Wong, Burlingame, CA (US); Peter Daddona, Menlo Park, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/911,299

(22) Filed: Aug. 3, 2004

Related U.S. Application Data
(60) Provisional application No. 60/492,610, filed on Aug. 4, 2003.

(51) Int. Cl. A61B 17/20; A61M 25/00

(52) U.S. Cl. 604/46; 604/264

(57) ABSTRACT

A substantially conical shaped microprojection having a wall defining an interior region, said microprojection wall including a plurality of openings; and a biocompatible coating disposed on the interior region of the microprojection. In a preferred embodiment, the microprojection is formed by deforming a region of a substantially planar sheet.

### CROSS REFERENCE TO RELATED APPLICATIONS

This application claims the benefit of U.S. Provisional Application No. 60/490,739, filed Aug. 4, 2003. Related applications include U.S. application Ser. No. ______, attorney docket number ALZ5037USANP, filed Aug. 3, 2004, and U.S. application Ser. No. ______, attorney docket number ALZ5037USANP2, filed Aug. 3, 2004.

### FIELD OF THE PRESENT INVENTION

The present invention relates generally to devices for transdermal delivery and sampling of agents. More particularly, this invention relates to the transdermal delivery of agents through a body surface, as well as the transdermal sampling of agents from a body surface, such as glucose, other body analytes and substances of abuse, such as alcohol and illicit drugs.

### BACKGROUND OF THE INVENTION

Interest in the transdermal delivery of beneficial agents, especially such agents as high molecular weight peptides, proteins and oligonucleotides and vaccines, to the human body by delivery across a body surface continues to grow as the number of such medically useful agents also grows and become available in large quantities and pure form. The terms “biologically active agent”, “agent”, “substance” and “drug” are used interchangeably herein and broadly include physiologically or pharmacologically active substances for producing a localized or systemic effect or effects in mammals, including humans and primates, avians, valuable domestic household, sport or farm animals, or for administering to laboratory animals, such as mice, rats, guinea pigs, and the like. The noted terms also include substances, such as glucose, other body analytes that are found in the tissue, interstitial fluid and/or blood, alcohol, licit substances, and illicit drugs, etc. that can be sampled through the skin.

Transdermal delivery of the noted agents still face significant problems. For example, in many instances, the rate of delivery or flux of such agents through the skin is insufficient to produce a desired therapeutic effect due to their large size/molecular weight and/or inability to pass through natural pathways (pores, hair follicles, etc.) that exist in the skin. Likewise, the passive flux of small (e.g., 200 to 500 daltons) water soluble agent molecules is often limited.

One method of increasing the transdermal delivery of agents is through the application of an electric current across the body surface, which is commonly referred to as “electrotransport”. As is well known in the art, “electrotransport” refers generally to the passage of a beneficial agent, e.g., a drug or drug precursor, through a body surface, such as skin, mucous membranes, nails, and the like. The transport of the agent is induced or enhanced by the application of an electrical potential, which results in the application of electric current that delivers or enhances delivery of the agent.

The electrotransport of agents through a body surface can be attained in various manners. One widely used electrotransport process, iontophoresis, involves the electrically induced transport of charged ions. Electroosmosis, another type of electrotransport process, involves the movement of a solvent with the agent through a membrane under the influence of an electric field. Electroporation, still another type of electrotransport, involves the passage of an agent through pores formed by applying a high voltage electrical pulse to a membrane. In many instances, more than one of these processes may be occurring simultaneously to different extents.

Accordingly, the term “electrotransport” is given herein its broadest possible interpretation, to include the electrically induced or enhanced transport of at least one charged or uncharged agent, or mixtures thereof, regardless of the specific mechanism(s) by which the agent is actually being transported.

Electrotransport delivery generally increases agent delivery, particularly large molecular weight species (e.g., polypeptides), relative to passive or non-electrically assisted transdermal delivery. However, further increases in transdermal delivery rates and reductions in polypeptide degradation during transdermal delivery are highly desirable.

One method of increasing the agent transdermal delivery rate involves pre-treating the skin with, or co-delivering with the beneficial agent, a skin permeation enhancer. The term “permeation enhancer” is broadly used herein to describe a substance which, when applied to a body surface through which the agent is delivered, enhances its flux therethrough. The mechanism may involve a reduction of the electrical resistance of the body surface to the passage of the agent therethrough, an increase in the permselectivity and/or permeability of the body surface, the creation of hydrophilic pathways through the body surface, and/or a reduction in the degradation of the agent (e.g., degradation by skin enzymes) during electrotransport.

There have also been many attempts to mechanically disrupt the skin in order to enhance transdermal flux, such as disclosed in U.S. Pat. No. 3,814,097 issued to Ganderton et al., U.S. Pat. No. 5,279,544 issued to Gross et al., U.S. Pat. No. 5,250,023 issued to Lee et al., U.S. Pat. No. 3,964,482 issued to Gerstel et al., U.S. Pat. No. Re 25,637 issued to Kravitz et al. and PCT Pub. No. WO 96/37155. The disclosed devices typically utilize tubular or cylindrical structures generally, although Gerstel does disclose the use of other shapes, to pierce the outer layer of the skin. The piercing elements disclosed in these references generally extend perpendicularly from a thin flat member, such as a pad or metal sheet.

More recently, attempts have been made to anchor the tiny piercing elements of such devices in the skin in order to keep the drug transmitting pathways open, which pathways are cut through the stratum corneum by the microprojections. See, for example, PCT Pub. No. WO 97/48440. Unfortunately, because of the extremely small size of the microprojections, the formation of barbs and similar anchoring elements on the microprojections is technically challenging and adds to the cost.

The microprojection arrays disclosed in PCT Pub. No. WO 97/48440 are in the form of a thin metal sheet having a plurality of agent-transmitting openings therethrough. The sheet has a skin proximal surface and a skin distal surface. A plurality of etched and punched microprojections extend roughly perpendicularly from the skin distal surface of the sheet. A reservoir adapted to contain (in the case of agent delivery) or receive (in the case of agent sampling) the agent is positioned on the skin distal surface of the sheet. The microprojection array and the agent reservoir are then pressed onto the skin surface and maintained on the skin using an adhesive overlay or similar securing means, as shown in FIG. 1 of Pub. No. WO 97/48440.

As illustrated in FIG. 1 and discussed in detail in the noted publication, sheet member 6, having the microprojections 4 extending from a skin distal surface thereof, is placed on the skin with the microprojections 4 penetrating into the skin surface. The agent reservoir 27 is shown on the skin distal side of sheet 6. The structure is held in place on the skin 30 by an overlay 3 having adhesive coated on at least the peripheral surfaces 9 thereof. In addition, the microprojections can be configured to include various skin retention elements, which also aid in retaining the microprojections within the skin.

The agent reservoir 27 of the device shown in FIG. 1 is generally composed of soft compliant materials such as gels. Such soft compliant, and even flowable, materials were preferred for use in conjunction with sheet member 6 since the gel material could easily flow into the openings of sheet member 6 in order to come into direct contact with skin 30.

As disclosed in U.S. patent application Ser. No. 10/045,842 and U.S. Pat. Pub. Nos. 2002/0193729, 2002/0177839 and 2002/0128599, which are fully incorporated by reference herein, it is possible to have the active agent that is to be delivered coated on the microprojections instead of contained in a physical reservoir. This eliminates the necessity of a separate physical reservoir and developing an agent formulation or composition specifically for the reservoir.

One drawback of coated microprojection systems is however the risk of physically displacing the coating from the microprojections during insertion of the microprojections into and through the skin (i.e., stratum corneum). As the microprojections are inserted into the skin, the skin tissue will push and rub up against the microprojections and any coating that has been placed thereon. It is thus possible to dislodge some or all of the coating whereby some or all of the coating is not inserted into the skin, not exposed to interstitial fluid and not dissolved and, hence, not made available for release into the skin.

A prior art example of microprojection array is shown in FIG. 1. Microprojection array 10 is composed of sheet 14 with microprojections 12 having been formed or etched out of sheet 14. The etching process or forming process forms microprojections 12 and openings 16. The microprojections 12 are then bent up and out of the plane of sheet 14.

As shown in FIG. 1, there are no surfaces on any of the microprojections 12 that are protected. If microprojection array 10 is placed upon and inserted into body surface, all faces of the microprojections 12 will be exposed to contact with the body surface and the underlying tissue. If the microprojections 12 have a coating 18 disposed thereon, as shown in FIG. 2, then such contact could dislodge and disrupt coatings 18.

This could result in a substantial amount of the agent not being deposited far enough into the tissue where it would be in contact with interstitial fluids. Without such contact, little, if any, of the agent in the coating would be released and be available to the recipient.

### SUMMARY OF THE INVENTION

The present invention substantially reduces or overcomes the limitations of prior art coated microprojection systems by transdermally delivering a biologically active agent using a microprojection array having a plurality of microprojections, at least one of the microprojections having an interior region that is coated with a solid, substantially dry coating containing at least one biologically active agent, wherein the microprojections can be inserted into and through the tissue (or stratum corneum) without substantially exposing the coating to physical contact with the tissue. The biologically active agent is selected to be sufficiently potent to be effective when delivered from a solid coating on a plurality of skin piercing microprojections. The coating preferably has sufficient water solubility such that when the microprojections are disposed within the patient's tissue the coating is easily and quickly dissolved, thereby releasing the biologically active agent.

One embodiment of this invention thus comprises a substantially conical shaped microprojection having a wall defining an interior region, said microprojection wall including a plurality of openings; and a biocompatible coating disposed on the interior region of the microprojection. In a preferred embodiment, the microprojection is formed by deforming (or extruding) a region of a substantially planar sheet.

In one embodiment of the invention, the microprojection is constructed out of a material selected from the group consisting of stainless steel, titanium, nickel titanium alloys and like biocompatible materials.

In another embodiment, the microprojection is constructed out of a non-conductive material.

In a further embodiment of the invention, the microprojection is coated with a non-conductive material.

In one embodiment of the invention, the microprojection has a length less than approximately 1000 microns.

Preferably, the biocompatible coating is produced by applying a coating formulation on the microprojection member.

In one embodiment of the invention, the coating formulation includes at least one biologically active agent selected from the group consisting of a hormone releasing hormone (LHRH), LHRH analog, vasopressin, desmopressin, corticotropin (ACTH), an ACTH analog, calcitonin, vasopressin, deamino [Val4, D-Arg8] arginine vasopressin, interferon alpha, interferon beta, interferon gamma, erythropoietin (EPO), granulocyte macrophage colony stimulating factor (GM-CSF), granulocyte colony stimulating factor (G-CSF), interleukin-10 (IL-10), glucagon, growth hormone releasing factor (GHRF), insulin, insulinotropin, calcitonin, octreotide, endorphin, TRN, NT-36 (chemical name: N-[[(s)-4-oxo-2-azetidinyl]carbonyl]-L-histidyl-L-prolinamide), liprecin, aANF, bMSH, somatostatin, bradykinin, somatotropin, platelet-derived growth factor releasing factor, chymopapain, cholecystokinin, chorionic gonadotropin, epoprostenol, hirulog, interferon, interleukin, menotropins, oxytocin, streptokinase, tissue plasminogen activator, urokinase, ANP, ANP clearance inhibitor, angiotensin II antagonist, antidiuretic hormone agonist, bradykinn antagonist, ceredase, CSI, calcitonin gene related peptide (CGRP), enkephalins, FAB fragment, IgE peptide suppressor, IGF-1, neurotrophic factor, colony stimulating factor, parathyroid hormone and agonist, parathyroid hormone antagonist, prostaglandin antagonist, pentigetide, protein C, protein S, renin inhibitor, thymosin alpha-1, thrombolytic, TNF, vasopressin antagonist analog, alpha-I antitrypsin (recombinant), TGF-beta, fondaparinux, ardeparin, dalteparin, defibrotide, enoxaparin, hirudin, nadroparin, reviparin, tinzaparin, pentosan polysulfate, oligonucleotide and oligonucleotide derivatives, alendronic acid, clodronic acid, etidronic acid, ibandronic acid, incadronic acid, pamidronic acid, risedronic acid, tiludronic acid, zoledronic acid, argatroban, RWJ 445167, and RWJ-671818.

In another embodiment of the invention, the coating formulation includes at least one vaccine selected from the group consisting of flu vaccine, Lyme disease vaccine, rabies vaccine, measles vaccine, mumps vaccine, chicken pox vaccine, small pox vaccine, hepatitis vaccine, pertussis vaccine, diphtheria vaccine, recombinant protein vaccine, DNA vaccine and therapeutic cancer vaccine.

In another embodiment of the invention, the coating formulation includes at least one buffer selected from the group consisting of ascorbic acid, citric acid, succinic acid, glycolic acid, gluconic acid, glucuronic acid, lactic acid, malic acid, pyruvic acid, tartaric acid, tartronic acid, fumaric acid, maleic acid, phosphoric acid, tricarballylic acid, malonic acid, adipic acid, citraconic acid, glutaratic acid, itaconic acid, mesaconic acid, citramalic acid, dimethylolpropionic acid, tiglic acid, glyceric acid, methacrylic acid, isocrotonic acid, crotonic acid, angelic acid, hydracrylic acid, aspartic acid, glutamic acid, glycine and mixtures thereof.

In another embodiment of the invention, the coating formulation includes at least one surfactant selected from the group consisting of sodium lauroamphoacetate, sodium dodecyl sulfate (SDS), cetylpyridinium chloride (CPC), dodecyltrimethyl ammonium chloride (TMAC), benzalkonium, chloride, polysorbates and other sorbitan derivatives.

In another embodiment of the invention, the coating formulation includes at least one polymeric material selected from the group consisting of hydroxyethylcellulose (HEC), hydroxypropylmethylcellulose (HPMC), hydroxypropycellulose (HPC), methylcellulose (MC), hydroxyethylmethylcellulose (HEMC) and ethylhydroxy-ethylcellulose (EHEC).

In another embodiment of the invention, the coating formulation includes at least one hydrophilic polymer selected from the group consisting of hyroxyethyl starch, dextran, poly(vinyl alcohol), poly(ethylene oxide), poly(2-hydroxyethylmethacrylate), poly(n-vinyl pyrolidone), polyethylene glycol and mixtures thereof.

In another embodiment of the invention, the coating formulation includes at least one biocompatible carrier selected from the group consisting of human albumin, bioengineered human albumin, polyglutamic acid, polyaspartic acid, polyhistidine, pentosan polysulfate, polyamino acids, sucrose, trehalose, melezitose, raffinose and stachyose.

In another embodiment of the invention, the coating formulation includes at least one stabilizing agent selected from the group consisting of a reducing sugar, non-reducing sugar and polysaccharide.

Preferably, the non-reducing sugar is selected from the group consisting of sucrose, trehalose, stachyose and raffinose.

Preferably, the polysaccharide is selected from the group consisting of dextran, soluble starch, dextrin and insulin.

Preferably, the reducing sugar is selected from the group consisting of apiose, arabinose, lyxose, ribose, xylose, digitoxose, fucose, quercitol, quinovose, rhamnose, allose, altrose, fructose, galactose, glucose, gulose, hamamelose, idose, mannose, tagatose, primeverose, vicianose, rutinose, scillabiose, cellobiose, gentiobiose, lactose, lactulose, maltose, melibiose, sophorose and turanose.

In another embodiment of the invention, the coating formulation includes at least one vasoconstrictor selected from the group consisting of amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and the mixtures thereof.

In yet another embodiment of the invention, the coating formulation includes at least one pathway patentency modulator selected from the group consisting of an osmotic agent, zwitterionic compound and anti-inflammatory agent.

Preferably, the anti-inflammatory agent is selected from the group consisting of betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate and prednisolone 21-succinate sodium salt.

In one embodiment of the invention, the pathway patentency modulator comprises an anticoagulant selected from the group consisting of citric acid, citrate salt, dextrin sulfate sodium, aspirin and EDTA.

In another embodiment of the invention, the coating formulation includes at least one solubilising/complexing agent selected from the group consisting of Alpha-Cyclodextrin, Beta-Cyclodextrin, Gamma-Cyclodextrin, glucosyl-alpha-Cyclodextrin, maltosyl-alpha-Cyclodextrin, glucosyl-beta-Cyclodextrin, maltosyl-beta-Cyclodextrin, hydroxypropyl beta-cyclodextrin, 2-hydroxypropyl-beta-Cyclodextrin, 2-hydroxypropyl-gamma-Cyclodextrin, hydroxyethyl-beta-Cyclodextrin, methyl-beta-Cyclodextrin, sulfobutylether-alpha-cyclodextrin, sulfobutylether-beta-cyclodextrin, and sulfobutylether-gamma-cyclodextrin. Most preferred solubilising/complexing agents are beta-cyclodextrin, hydroxypropyl beta-cyclodextrin, 2-hydroxypropyl-beta-Cyclodextrin and sulfobutylether7 beta-cyclodextrin.

In a preferred embodiment, the coating formulation has a viscosity less than approximately 500 centipoise and greater than 3 centipose.

Preferably, the coating has a thickness less than 100 microns.

### BRIEF DESCRIPTION OF THE DRAWINGS

FIG. 1 is a perspective view of a prior art microprojection array that does not incorporate any protective features;

FIG. 2 is a perspective view of a prior art microprojection array that is similar to the array shown in FIG. 1, having an agent coating;

FIG. 3A is a perspective view of an embodiment of the present invention wherein the microprojection has a standard hollow needle-like configuration and a longitudinal slit;

FIG. 3B is a perspective view of an embodiment of the present invention wherein the microprojection has a standard hollow needle-like configuration and a plurality of perforations that extend through the walls;

FIG. 3C is a perspective view of an embodiment of the present invention wherein the microprojection comprises a porous ceramic material having a standard hollow needle-like configuration;

FIG. 3D is a perspective view of another embodiment of the present invention wherein the microprojection comprises a porous ceramic material having a standard hollow needle-like configuration;

FIG. 4 is a top plane view of a sheet, illustrating a plurality of microprojections that have been etched out of the sheet and prior to the microprojections being bent perpendicular to the sheet according to the invention;

FIG. 5 is a perspective view of the sheet shown in FIG. 4 wherein the microprojections have been bent substantially perpendicular to the plane of the sheet according to the invention;

FIG. 6 is a top plane view of another flat sheet, illustrating a plurality of microprojections having slits etched into the body of the microprojections according to the invention;

FIG. 7 is a perspective view of the sheet shown in FIG. 6 wherein the microprojections have been bent substantially perpendicular to the plane of the sheet according to the invention;

FIG. 8 is a perspective view of an embodiment of the present invention that is similar to the embodiment shown in FIG. 5, but which also includes a supporting brace attached between the tips of each pair of microprojections;

FIG. 9 is a perspective view of an embodiment of the present invention, similar to the embodiment shown in FIG. 7, but which also includes a supporting brace attached between the tips of each pair of microprojections;

FIG. 10A is a plane view of an embodiment of the present invention, which shows a flat sheet having a plurality of groups of small holes etched into the flat sheet; and

FIG. 10B is a perspective view of the flat sheet shown in FIG. 10A after the sheet has been modified to form a plurality of microprojections centered around the groupings of small holes.

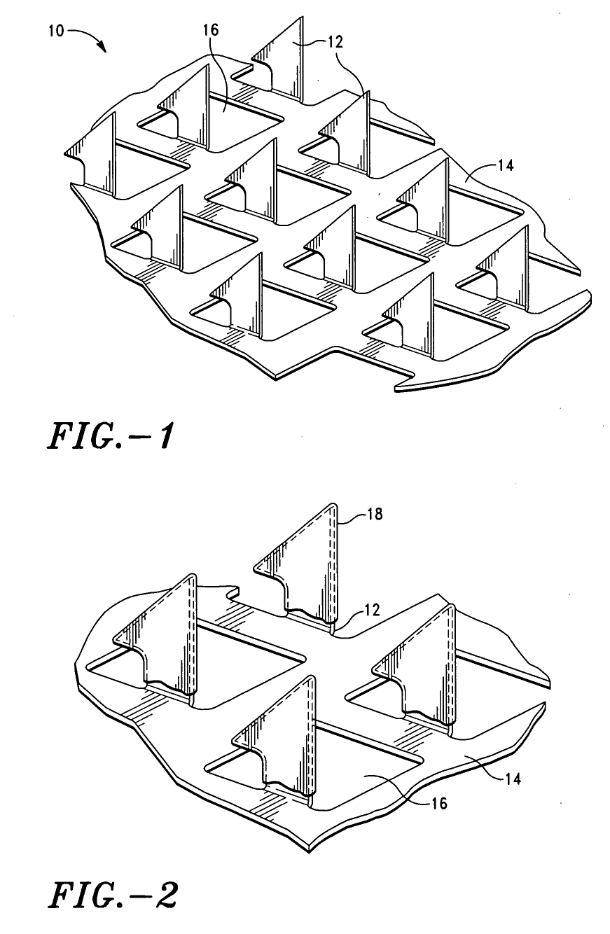

### DETAILED DESCRIPTION OF THE INVENTION

Before describing the present invention in detail, it is to be understood that this invention is not limited to particularly exemplified materials, methods or structures as such may, of course, vary. Thus, although a number of materials and methods similar or equivalent to those described herein can be used in the practice of the present invention, the preferred materials and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “an active agent” includes two or more such agents; reference to “a microprojection” includes two or more such microprojections and the like.

### Definitions

The term “body surface”, as used herein, refers generally to the skin, mucous membranes, and nails of an animal or human, and to the outer surface of a plant.

The term “transdermal”, as used herein, means the delivery of an agent into and/or through the skin for local or systemic therapy.

The term “transdermal flux”, as used herein, means the rate of transdermal delivery.

The term “co-delivering”, as used herein, means that a supplemental agent(s) is administered transdermally either before the agent is delivered, before and during transdermal flux of the agent, during transdermal flux of the agent, during and after transdermal flux of the agent, and/or after transdermal flux of the agent. Additionally, two or more biologically active agents may be formulated in the coating formulations of the invention, resulting in co-delivery of the biologically active agents.

The terms “biologically active agent” and “agent”, as used herein, refer to a composition of matter or mixture containing a drug that is pharmacologically effective when administered in a therapeutically effective amount. Examples of such active agents include, without limitation, small molecular weight compounds, polypeptides, proteins, oligonucleotides, nucleic acids and polysaccharides.

Further examples of “biologically active agents” include, without limitation, leutinizing hormone releasing hormone (LHRH), LHRH analogs (such as goserelin, leuprolide, buserelin, triptorelin, gonadorelin, and napfarelin, menotropins (urofollitropin (FSH) and LH)), vasopressin, desmopressin, corticotropin (ACTH), ACTH analogs such as ACTH (1-24), calcitonin, vasopressin, deamino [Val4, D-Arg8] arginine vasopressin, interferon alpha, interferon beta, interferon gamma, erythropoietin (EPO), granulocyte macrophage colony stimulating factor (GM-CSF), granulocyte colony stimulating factor (G-CSF), interleukin-10 (IL-10), glucagon, growth hormone releasing factor (GHRF), insulin, insulinotropin, calcitonin, octreotide, endorphin, TRN, NT-36 (chemical name: N-[[(s)-4-oxo-2-azetidinyl]carbonyl]-L-histidyl-L-prolinamide), liprecin, aANF, bMSH, somatostatin, bradykinin, somatotropin, platelet-derived growth factor releasing factor, chymopapain, cholecystokinin, chorionic gonadotropin, epoprostenol (platelet aggregation inhibitor), glucagon, hirulog, interferons, interleukins, menotropins (urofollitropin (FSH) and LH), oxytocin, streptokinase, tissue plasminogen activator, urokinase, ANP, ANP clearance inhibitors, angiotensin II antagonists, antidiuretic hormone agonists, bradykinn antagonists, ceredase, CSI's, calcitonin gene related peptide (CGRP), enkephalins, FAB fragments, IgE peptide suppressors, IGF-1, neurotrophic factors, colony stimulating factors, parathyroid hormone and agonists, parathyroid hormone antagonists, prostaglandin antagonists, pentigetide, protein C, protein S, renin inhibitors, thymosin alpha-1, thrombolytics, TNF, vasopressin antagonists analogs, alpha-1 antitrypsin (recombinant), TGF-beta, fondaparinux, ardeparin, dalteparin, defibrotide, enoxaparin, hirudin, nadroparin, reviparin, tinzaparin, pentosan polysulfate, oligonucleotides and oligonucleotide derivatives such as formivirsen, alendronic acid, clodronic acid, etidronic acid, ibandronic acid, incadronic acid, pamidronic acid, risedronic acid, tiludronic acid, zoledronic acid, argatroban, RWJ 445167, and RWJ-671818.

The noted biologically active agents can also be in various forms, such as free bases, acids, charged or uncharged molecules, components of molecular complexes or nonirritating, pharmacologically acceptable salts. Further, simple derivatives of the active agents (such as ethers, esters, amides, etc.), which are easily hydrolyzed at body pH, enzymes, etc., can be employed.

The term “biologically active agent”, as used herein, also refers to a composition of matter or mixture containing a “vaccine” or other immunologically active agent or an agent which is capable of triggering the production of an immunologically active agent, and which is directly or indirectly immunologically effective when administered in an immunologically effective amount.

The term “vaccine”, as used herein, refers to conventional and/or commercially available vaccines, including, but not limited to, flu vaccines, Lyme disease vaccine, rabies vaccine, measles vaccine, mumps vaccine, chicken pox vaccine, small pox vaccine, hepatitis vaccine, pertussis vaccine, diphtheria vaccine, recombinant protein vaccines, DNA vaccines and therapeutic cancer vaccines. The term “vaccine” thus includes, without limitation, antigens in the form of proteins, polysaccharides, oligosaccharides, lipoproteins, weakened or killed viruses such as cytomegalovirus, hepatitis B virus, hepatitis C virus, human papillomavirus, rubella virus, and varicella zoster, weakened or killed bacteria such as bordetella pertussis, clostridium tetani, corynebacterium diphtheriae, group A streptococcus, legionella pneumophila, neisseria meningitides, pseudomonas aeruginosa, streptococcus pneumoniae, treponema pallidum, and vibrio cholerae and mixtures thereof.

It is to be understood that more than one biologically active agent may be incorporated into the coating formulations and coatings produced therefrom of this invention, and that the use of the term “biologically active agent” (or “active agent”) in no way excludes the use of two or more such active agents.

The term “biologically effective amount” or “biologically effective rate” shall be used when the biologically active agent is a pharmaceutically active agent and refers to the amount or rate of the pharmacologically active agent needed to effect the desired therapeutic, often beneficial, result. The amount of active agent employed in the coatings of the invention will be that amount necessary to deliver a therapeutically effective amount of the active agent to achieve the desired therapeutic result. In practice, this will vary widely depending upon the particular pharmacologically active agent being delivered, the site of delivery, the severity of the condition being treated, the desired therapeutic effect and the release kinetics for delivery of the agent from the coating into skin tissues.

The term “biologically effective amount” or “biologically effective rate” shall also be used when the biologically active agent is an immunologically active agent and refers to the amount or rate of the immunologically active agent needed to stimulate or initiate the desired immunologic, often beneficial result. The amount of the immunologically active agent employed in the coatings of the invention will be that amount necessary to deliver an amount of the active agent needed to achieve the desired immunological result. In practice, this will vary widely depending upon the particular immunologically active agent being delivered, the site of delivery, and the dissolution and release kinetics for delivery of the active agent into skin tissues.

The terms “agent” and “substance”, as used herein, also include substances, such as glucose, other body analytes that are found in the tissue, interstitial fluid and/or blood, alcohol, licit substances, and illicit drugs, etc. that can be sampled through the skin.

The term “microprojections”, as used herein, refers to piercing elements that are adapted to pierce or cut through the stratum corneum into the underlying epidermis layer, or epidermis and dermis layers, of the skin of a living animal, particularly a mammal and more particularly a human. The term “microprojection” thus includes such projections often referred to as microblades, lances, microneedles, etc.

As discussed in detail herein, in one embodiment of the invention, the microprojections preferably have a projection length of less than 1000 microns, more preferably, less than 250 microns.

The term “microprojection array”, as used herein, refers to a plurality of microprojections arranged in an array for piercing the stratum corneum. As discussed in detail herein, the microprojection array can be formed by etching or punching a plurality of microprojections from a thin sheet and folding or bending the microprojections out of the plane of the sheet to form a configuration.

The terms “biocompatible coating” and “coating”, as used herein, refer to a composition that is employed to coat the microprojections. In at least one embodiment of the invention, the coating includes at least one active agent therein and, optionally, a biocompatible carrier. According to the invention, the coating is selected for its adhesion properties, its stabilization properties, its ability to be quickly dissolved within the epidermis layer, and its ability to form a structure that retains soluble agents and insoluble agents when substantially dried on the microprojections.

As indicated above, in one embodiment, the present invention comprises a device for forming a microslit through the stratum corneum for transdermally delivering a biologically active agent into and through the stratum corneum or sampling an agent through the stratum corneum, the device including a microprojection member having exterior and interior regions, the interior region having a biocompatible coating disposed thereon, the coating including at least one agent, the microprojection member being adapted to substantially restrict contact of the coating with the stratum corneum during insertion of the microprojection into the stratum corneum.

In another embodiment of the invention, the device comprises a plurality of microprojections, each of the microprojections having an interior region that is coated with a solid, substantially dry coating containing at least one biologically active agent, wherein the microprojections can be inserted into and through the tissue (or stratum corneum) without substantially exposing the coating to physical contact with the tissue.

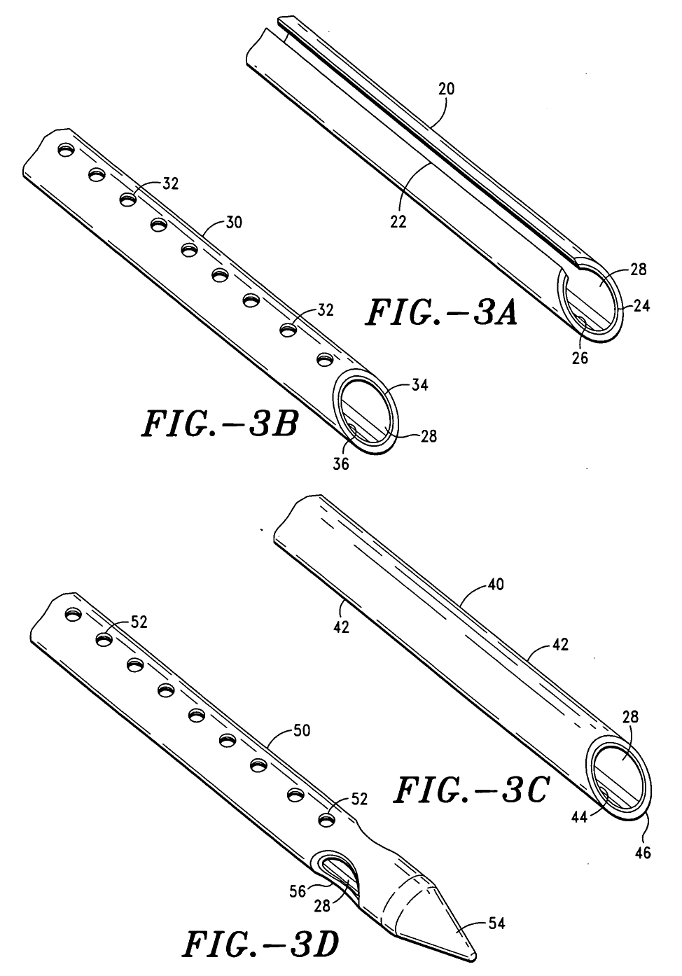

Referring now to FIG. 3A, there is shown one embodiment of a microprojection 20 that can be employed within the scope of the present invention. As illustrated if FIG. 3A, the microprojection 20 has a shape that is similar to a standard hollow syringe needle. The microprojection 20 also includes a slit 22 that extends rearward from the tip 24. According to the invention, the slit 22 can extend partially or fully over the length of the microprojection 20.

In a preferred embodiment, the slit 22 extends longitudinally, as shown in FIG. 3A, and is preferably disposed substantially parallel to the longitudinal axis of the microprojection 20. In additional embodiments, not shown, the slit 22 can extend spirally or substantially perpendicular to the longitudinal axis. In the noted embodiments, more than one slit can also be employed.

According to the invention, a coating formulation (discussed in detail below) is disposed on the interior region 26 of the microprojection 20 and dried to form a solid coating 28. When the coated microprojection 20 is inserted into the skin (i.e., into and/or through the stratum corneum), contact of the skin and underlying tissue with the coating is substantially restricted; the slit 22 providing means by which interstitial fluid from the surrounding tissue can come in contact with the coating 28, thereby dissolving the coating 28 and releasing any agent disposed therein.

Referring now to FIG. 3B, there is shown another embodiment of a microprojection 30 of the invention. As illustrated in FIG. 3B, the microprojection 30 has a shape similar to microprojection 20 shown in FIG. 3A. However, in this embodiment, instead of a slit, the microprojection 30 includes a plurality of perforations 32 that extend through the wall 34 of the microprojection 30.

As illustrated in FIG. 3B, the interior region 36 is similarly coated with a coating formulation to form a solid coating 28. According to the invention, when the coated microprojection 30 is inserted into the skin, contact with the skin and underlying tissue with the coating is similarly substantially restricted; the perforations 32 in the wall 34 of the microprojection 30 providing means by which interstitial fluid from the surrounding tissue can come in contact with the coating 28, thereby dissolving the coating 28 and releasing any agent disposed therein.

In one embodiment, the microprojections 20, 30 are constructed out of stainless steel, titanium, nickel titanium alloys, or similar biocompatible materials.

In another embodiment, the microprojections 20, 30 are constructed out of a non-conductive material, such as a polymer. Alternatively, the microprojections 20, 30 can be coated with a non-conductive material, such as Parylene®, or a hydrophobic material, such as Teflon®, silicon or other low energy material.

Preferably, the microprojections 20, 30 have a length less than approximately 1000 microns, more preferably, less than approximately 500 microns and an outer diameter in the range of approximately 20-200 microns.

According to the invention, the coating formulations applied to the microprojections 20, 30 to form the solid biocompatible coating 28 can comprise aqueous and non-aqueous formulations.

In at least one embodiment, the biocompatible coating 28 includes at least one biologically active agent which can comprise, without limitation, leutinizing hormone releasing hormone (LHRH), LHRH analogs (such as goserelin, leuprolide, buserelin, triptorelin, gonadorelin, and napfarelin, menotropins (urofollitropin (FSH) and LH)), vasopressin, desmopressin, corticotropin (ACTH), ACTH analogs such as ACTH (1-24), calcitonin, vasopressin, deamino [Val4, D-Arg8] arginine vasopressin, interferon alpha, interferon beta, interferon gamma, erythropoietin (EPO), granulocyte macrophage colony stimulating factor (GM-CSF), granulocyte colony stimulating factor (G-CSF), interleukin-10 (IL-10), glucagon, growth hormone releasing factor (GHRF), insulin, insulinotropin, calcitonin, octreotide, endorphin, TRN, NT-36 (chemical name: N-[[(s)-4-oxo-2-azetidinyl]carbonyl]-L-histidyl-L-prolinamide), liprecin, aANF, bMSH, somatostatin, bradykinin, somatotropin, platelet-derived growth factor releasing factor, chymopapain, cholecystokinin, chorionic gonadotropin, epoprostenol (platelet aggregation inhibitor), glucagon, hirulog, interferons, interleukins, menotropins (urofollitropin (FSH) and LH), oxytocin, streptokinase, tissue plasminogen activator, urokinase, ANP, ANP clearance inhibitors, angiotensin II antagonists, antidiuretic hormone agonists, bradykinn antagonists, ceredase, CSI's, calcitonin gene related peptide (CGRP), enkephalins, FAB fragments, IgE peptide suppressors, IGF-1, neurotrophic factors, colony stimulating factors, parathyroid hormone and agonists, parathyroid hormone antagonists, prostaglandin antagonists, pentigetide, protein C, protein S, renin inhibitors, thymosin alpha-1, thrombolytics, TNF, vasopressin antagonists analogs, alpha-1 antitrypsin (recombinant), TGF-beta, fondaparinux, ardeparin, dalteparin, defibrotide, enoxaparin, hirudin, nadroparin, reviparin, tinzaparin, pentosan polysulfate, oligonucleotides and oligonucleotide derivatives such as formivirsen, alendronic acid, clodronic acid, etidronic acid, ibandronic acid, incadronic acid, pamidronic acid, risedronic acid, tiludronic acid, zoledronic acid, argatroban, RWJ 445167, and RWJ-671818.

The biologically active agent can further include conventional and/or commercially available vaccines, including, but not limited to, flu vaccines, Lyme disease vaccine, rabies vaccine, measles vaccine, mumps vaccine, chicken pox vaccine, small pox vaccine, hepatitis vaccine, pertussis vaccine, and diphtheria vaccine, recombinant protein vaccines, DNA vaccines and therapeutic cancer vaccines, e.g., antigens in the form of proteins, polysaccharides, oligosaccharides, lipoproteins, weakened or killed viruses such as cytomegalovirus, hepatitis B virus, hepatitis C virus, human papillomavirus, rubella virus, and varicella zoster, weakened or killed bacteria such as bordetella pertussis, clostridium tetani, corynebacterium diphtheriae, group A streptococcus, legionella pneumophila, neisseria meningitides, pseudomonas aeruginosa, streptococcus pneumoniae, treponema pallidum, and vibrio cholerae and mixtures thereof.

In one embodiment of the invention, the coating formulation includes at least one buffer. Examples of such buffers include ascorbic acid, citric acid, succinic acid, glycolic acid, gluconic acid, glucuronic acid, lactic acid, malic acid, pyruvic acid, tartaric acid, tartronic acid, fumaric acid, maleic acid, phosphoric acid, tricarballylic acid, malonic acid, adipic acid, citraconic acid, glutaratic acid, itaconic acid, mesaconic acid, citramalic acid, dimethylolpropionic acid, tiglic acid, glyceric acid, methacrylic acid, isocrotonic acid, □-hydroxybutyric acid, crotonic acid, angelic acid, hydracrylic acid, aspartic acid, glutamic acid, glycine or mixtures thereof.

In one embodiment of the invention, the coating formulation includes at least one surfactant, which can be zwitterionic, amphoteric, cationic, anionic, or nonionic, including, without limitation, sodium lauroamphoacetate, sodium dodecyl sulfate (SDS), cetylpyridinium chloride (CPC), dodecyltrimethyl ammonium chloride (TMAC), benzalkonium, chloride, polysorbates such as Tween 20 and Tween 80, other sorbitan derivatives, such as sorbitan laurate, and alkoxylated alcohols, such as laureth-4.

In a further embodiment of the invention, the coating formulation includes at least one polymeric material or polymer that has amphiphilic properties, which can comprise, without limitation, cellulose derivatives, such as hydroxyethylcellulose (HEC), hydroxypropylmethylcellulose (HPMC), hydroxypropycellulose (HPC), methylcellulose (MC), hydroxyethylmethylcellulose (HEMC), or ethylhydroxy-ethylcellulose (EHEC), as well as pluronics.

In another embodiment, the coating formulation includes a hydrophilic polymer selected from the following group: hyroxyethyl starch, dextran, poly(vinyl alcohol), poly(ethylene oxide), poly(2-hydroxyethylmethacrylate), poly(n-vinyl pyrolidone), polyethylene glycol and mixtures thereof, and like polymers.

In another embodiment of the invention, the coating formulation includes a biocompatible carrier, which can comprise, without limitation, human albumin, bioengineered human albumin, polyglutamic acid, polyaspartic acid, polyhistidine, pentosan polysulfate, polyamino acids, sucrose, trehalose, melezitose, raffinose and stachyose.

In another embodiment, the coating formulation includes a stabilizing agent, which can comprise, without limitation, a non-reducing sugar, a polysaccharide or a reducing sugar. Suitable non-reducing sugars for use in the methods and compositions of the invention include, for example, sucrose, trehalose, stachyose, or raffinose. Suitable polysaccharides for use in the methods and compositions of the invention include, for example, dextran, soluble starch, dextrin, and insulin. Suitable reducing sugars for use in the methods and compositions of the invention include, for example, monosaccharides such as, for example, apiose, arabinose, lyxose, ribose, xylose, digitoxose, fucose, quercitol, quinovose, rhamnose, allose, altrose, fructose, galactose, glucose, gulose, hamamelose, idose, mannose, tagatose, and the like; and disaccharides such as, for example, primeverose, vicianose, rutinose, scillabiose, cellobiose, gentiobiose, lactose, lactulose, maltose, melibiose, sophorose, and turanose, and the like.

In another embodiment, the coating formulation includes a vasoconstrictor, which can comprise, without limitation, amidephrine, cafaminol, cyclopentamine, deoxyepinephrine, epinephrine, felypressin, indanazoline, metizoline, midodrine, naphazoline, nordefin, octodrine, omipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and the mixtures thereof. The most preferred vasoconstrictors include epinephrine, naphazoline, tetrahydrozoline indanazoline, metizoline, tramazoline, tymazoline, oxymetazoline and xylometazoline.

In another embodiment of the invention, the coating formulation includes at least one “pathway patency modulator”, which can comprise, without limitation, osmotic agents (e.g., sodium chloride), zwitterionic compounds (e.g., amino acids), and anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate and prednisolone 21-succinate sodium salt, and anticoagulants, such as citric acid, citrate salts (e.g., sodium citrate), dextrin sulfate sodium, aspirin and EDTA.

In yet another embodiment of the invention, the coating formulation includes a solubilising/complexing agent, which can comprise Alpha-Cyclodextrin, Beta-Cyclodextrin, Gamma-Cyclodextrin, glucosyl-alpha-Cyclodextrin, maltosyl-alpha-Cyclodextrin, glucosyl-beta-Cyclodextrin, maltosyl-beta-Cyclodextrin, hydroxypropyl beta-cyclodextrin, 2-hydroxypropyl-beta-Cyclodextrin, 2-hydroxypropyl-gamma-Cyclodextrin, hydroxyethyl-beta-Cyclodextrin, methyl-beta-Cyclodextrin, sulfobutylether-alpha-cyclodextrin, sulfobutylether-beta-cyclodextrin, and sulfobutylether-gamma-cyclodextrin. Most preferred solubilising/complexing agents are beta-cyclodextrin, hydroxypropyl beta-cyclodextrin, 2-hydroxypropyl-beta-Cyclodextrin and sulfobutylether7 beta-cyclodextrin.

In another embodiment of the invention, the coating formulation includes at least one non-aqueous solvent, such as ethanol, isopropanol, methanol, propanol, butanol, propylene glycol, dimethysulfoxide, glycerin, N,N-dimethylformamide and polyethylene glycol 400.

Preferably, the coating formulations have a viscosity less than approximately 500 centipoise and greater than 3 centipose.

In one embodiment of the invention, the thickness of the biocompatible coating is less than 100 microns, more preferably, less than 50 microns, as measured from the microprojection surface.

Referring now to FIG. 3C, there is shown another embodiment of a microprojection 40 of the invention. According to the invention, the microprojection 40 has a similar shape and size as the microprojections 20, 30 shown in FIGS. 3A and 3B. However, in this embodiment, the microprojection 40 is formed from a ceramic or like material. Preferably, the ceramic material exhibits a high surface energy and has a total porosity in the range of approximately 10-80%.

In one embodiment of the invention, the ceramic material has an average pore size in the range of approximately 0.5-50 microns. In the embodiment shown in FIG. 3C, the noted porosity is facilitated (or enhanced) via a plurality of slits 42.

As will be appreciated by one having ordinary skill in the art, the desired porosity can also be achieved by other conventional fabrication means. As will further be appreciated by on having ordinary skill in the art, the porosity and/or pore size characteristics of the ceramic material used in the fabrication of the ceramic microprojections can be selected based on the coating formulation employed and/or the molecular characteristics of the particular agent being delivered.

As illustrated in FIG. 3C, the interior region 44 of the microprojection 40 is similarly coated with a coating formulation to form a solid coating 28. According to the invention, when the coated microprojection 40 is inserted into the skin, contact with the skin and underlying tissue with the coating is similarly substantially restricted; the porous ceramic material providing means by which interstitial fluid from the surrounding tissue can come in contact with the coating 28, thereby dissolving the coating 28 and releasing any agent disposed therein. The released agent will then diffuse out from the interior region 44 of the microprojection 40, either back through the porous ceramic wall or through the opening 46 at the end of the microprojection 40.

According to the invention, the coating formulation applied the microprojection 40 to from the solid coating can similarly comprise any of the aforementioned coating formulations. The active agent can similarly comprise any of the aforementioned agents.

Referring now to FIG. 3D, there is shown yet another embodiment of a microprojection 50 of the invention, which is similarly preferably formed from a porous ceramic material. According to the invention, the microprojection 50 has a similar shape and size as microprojection 30, shown in FIG. 3B, including a plurality of perforations 52. However, in this embodiment, the microprojection 50 includes a solid piercing edge 54 and one or more openings 56 disposed proximate the piercing edge 54 to aid in the dissolution of the coating 28 disposed in the interior region of the microprojection 50.

According to the invention, openings 56 can comprise various shapes and sizes to achieve the desired introduction of interstitial fluid(s) and release of the agent(s) contained in the coating. In a preferred embodiment, the openings 56 have a curvilinear or scalloped shape.

As illustrated in FIG. 3D, the interior region of the microprojection 50 is similarly coated with a coating formulation to form a solid coating 28. According to the invention, when the coated microprojection 50 is inserted into the skin, contact with the skin and underlying tissue with the coating is similarly substantially restricted; the perforations 52, openings 56 and porous ceramic material providing means by which interstitial fluid from the surrounding tissue can come in contact with the coating 28, thereby dissolving the coating 28 and releasing any agent disposed therein. The agent will then diffuse out from the interior region of the microprojection 50, either back through the perforations 52, openings 56 or porous ceramic wall of the microprojection 50.

According to the invention, the coating formulation applied the interior region of the microprojection 50 to from the solid coating can similarly comprise any of the aforementioned coating formulations. The active agent can similarly comprise any of the aforementioned agents.

Preferably, the microprojections 40, 50 have a length less than approximately 1000 microns, more preferably, less than approximately 500 microns and an outer diameter in the range of approximately 20-200 microns.

Referring now to FIG. 4, there is shown the first phase in the manufacture of a second general embodiment of the invention. A microprojection array 60A is initially formed from a thin sheet 61 by etching away material to provide openings 68. As illustrated in FIG. 4, proximate the etched openings 68 are microprojections 62 and 64. At this stage, the microprojections 62 and 64 are still positioned in the plane of sheet 61.

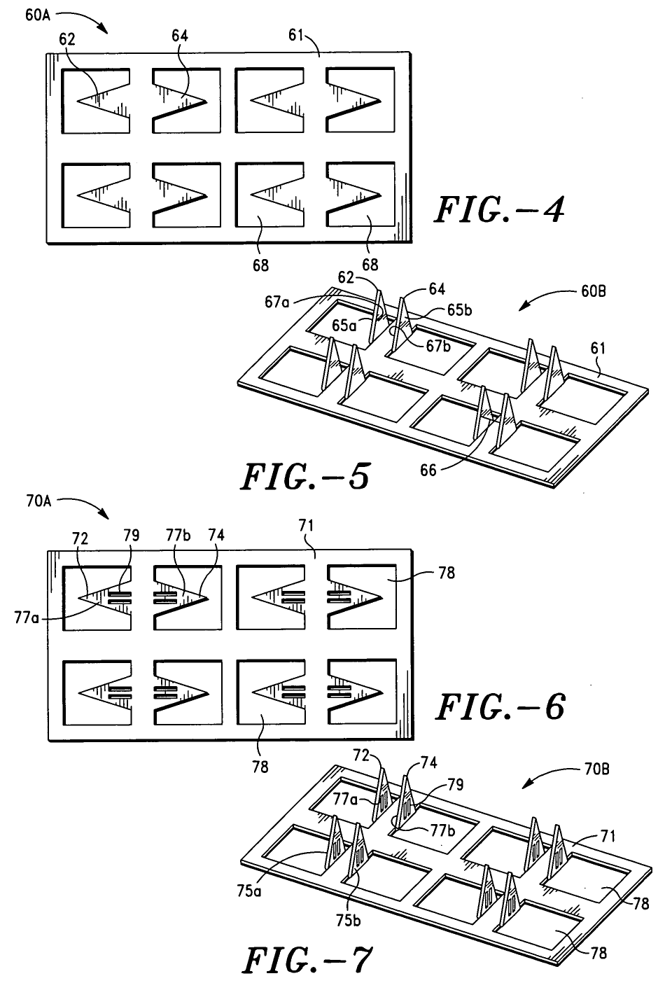

Referring now to FIG. 5, there is shown the microprojection array 60B with the microprojections 62 and 64 bent out of the plane of sheet 61 and separated from each other by gap 66. As illustrated in FIG. 5, the microprojections 62, 64 are preferably bent substantially perpendicular to the sheet 61 and are disposed substantially parallel to each other. As further illustrated in FIG. 5, the microprojections 62 and 64 include inner faces 67a, 67b, which face each other, and outer surfaces 65a, 65b.

In a preferred embodiment of the invention, after the microprojections 62, 64 are bent out of the sheet 61, a coating formulation is applied to at least one, preferably, both of the inner surfaces 67a, 67b of the microprojections 62, 64 to form a solid coating. According to the invention, the coating is protected from being dislodged or abraded as the microprojections 62, 64 are inserted into the skin.

In a further embodiment of the invention, the coating formulation is applied to each microprojection 62 and 64 prior to the microprojections 62, 64 being bent out of the plane of the sheet 61.

In a further envisioned embodiment of the invention, the coating formulation is also applied to the outer surfaces 65a, 65b of the microprojections 62, 64 to form an additional coating thereon.

Referring now to FIGS. 6 and 7, there is shown the formation of a further embodiment of a microprojection array of the invention. As illustrated in FIG. 6, the microprojection array 70A is similarly formed by etching openings 78 in a thin sheet of material 71. Disposed proximate the openings 78 are microprojections 72, 74.

Referring now to FIG. 7, the microprojections 72, 74 are similarly bent substantially perpendicular to the plane of the sheet 71 with inner surfaces 77a, 77b facing each other. As illustrated in FIGS. 6 and 7, each microprojection 72, 74 includes at least one, preferably, a plurality of openings 79 that are disposed in the body of each microprojection 72, 74.

According to the invention, the openings 79 can comprise various shapes and sizes. In a preferred embodiment, the openings are substantially rectangular in shape.

In a preferred embodiment of the invention, after the microprojections 72, 74 are bent out of the sheet 71, a coating formulation is similarly applied to at least one, preferably, both of the inner surfaces 77a, 77b of the microprojections 72 and 74 to form a solid coating. In a further embodiment of the invention, the coating formulation is applied to each microprojection 72 and 74 prior to the microprojections 72, 74 being bent out of the plane of the sheet 71.

According to the invention, the openings 79 facilitate the contact of interstitial fluid of the body with the coating after the microprojection array 70B has been inserted into the skin. The openings 79 further facilitate the dissolution of the coating in the protected space between the microprojections 72, 74 that is defined by the inner surfaces 77a, 77b and the release of the agent-containing coating into the body.

In a further envisioned embodiment of the invention, the coating formulation is also applied to the outer surfaces 75a, 75b of the microprojections 72, 74 to form an additional coating thereon.

Referring now to FIG. 8, there is shown another embodiment of a microprojection array 60C of the invention. As illustrated in FIG. 8, the microprojection array 60C is similar to array 60B shown in FIG. 5. However, in this embodiment, the array 60C includes a brace 80, which is preferably affixed the tips of microprojections 62 and 64. According to the invention, brace 80 provides additional structural rigidity and assists in maintaining the distance between the inner surfaces 67a, 67b between the microprojections 62, 64 (i.e., gap 66).

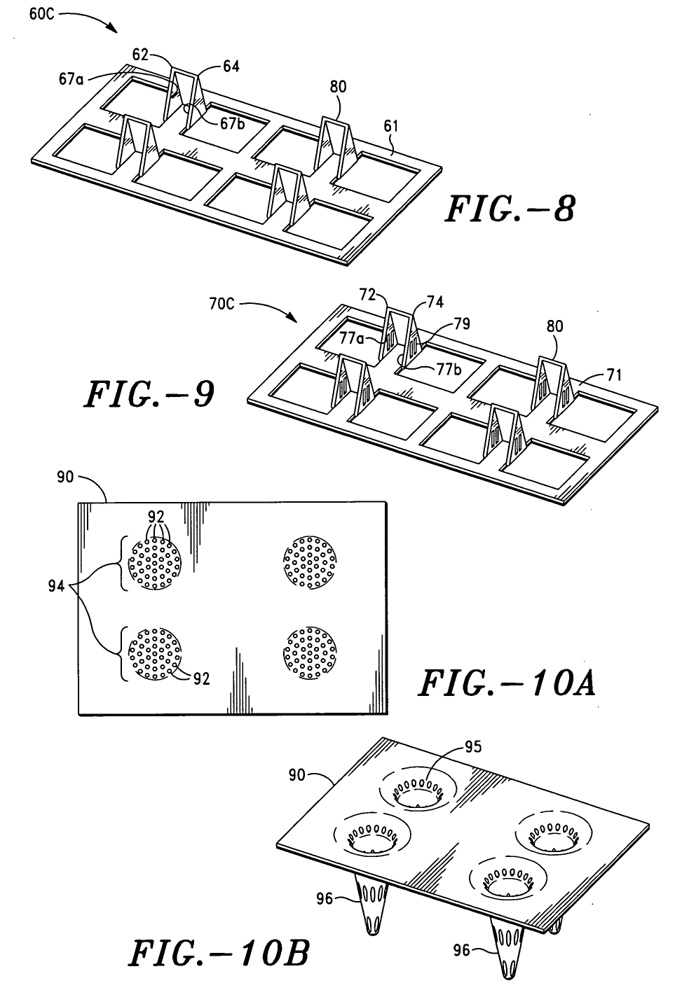

Referring now to FIG. 9, there is shown yet another embodiment of a microprojection array 70C of the invention. As illustrated in FIG. 9, the microprojection array 70C is similar to array 70B shown in FIG. 7 and similarly includes brace 80, which is preferably affixed the tips of microprojections 72 and 74.

The gap 66 between the microprojections 62, 64 and 72, 74 is preferably sized such that the pair of microprojections (e.g. 62, 64) act as a single penetration device and that there is no “coring”, i.e., there is no insertion of tissue between the microprojections as the microprojections are inserted into the skin. Typically, the gap 66 between respective pairs of microprojections is in the range of approximately 25 microns to 250 microns.

Preferably, the microprojections 62, 64, 72, 74 have a length less than approximately 1000 microns, more preferably, less than approximately 500 microns.

In a preferred embodiment of the invention, the microprojections 62, 64, 72, 74 are constructed out of stainless steel, titanium, nickel titanium alloys, or a similar biocompatible material. Alternatively, the microprojections 62, 64, 72, 74 can be coated with a non-conductive material, such as Parylene®, or a hydrophobic material, such as Teflon®, silicon or other low energy material.

In a further envisioned embodiment, the microprojections 62, 64, 72, 74 are formed from a non-conductive material, such as a polymer.

According to the invention, the coating formulation can be applied to the microprojections 62, 64, 72, 74 by a variety of known methods. One such coating method comprises dip-coating. Dip-coating can be described as a means to coat the microprojections by partially or totally immersing the microprojections 62, 64, 72, 74 into a coating solution. By use of a partial immersion technique, it is possible to limit the coating to only the tips of the microprojections 62, 64, 72, 74.

A further coating method comprises roller coating, which employs a roller coating mechanism that similarly limits the coating to the tips of the microprojections 62, 64, 72, 74. The roller coating method is disclosed in U.S. application Ser. No. 10/099,604 (Pub. No. 2002/0132054), which is incorporated by reference herein in its entirety. As discussed in detail in the noted application, the roller coating method provides a smooth coating that further restricts the coating from being dislodged from the microprojections 62, 64, 72, 74 during skin piercing.

According to the invention, the microprojections 62, 64, 72, 74 can further include means adapted to receive and/or enhance the volume of the coating 35, such as grooves (not shown), surface irregularities (not shown) or similar modifications, wherein the means provides increased surface area upon which a greater amount of coating can be deposited.

A further coating method that can be employed within the scope of the present invention comprises spray coating. According to the invention, spray coating can encompass formation of an aerosol suspension of the coating composition.

Pattern coating can also be employed to coat the microprojections 62, 64, 72, 74. The pattern coating can be applied using a dispensing system for positioning the deposited liquid onto the microprojection surface. Examples of suitable precision-metered liquid dispensers are disclosed in U.S. Pat. Nos. 5,916,524; 5,743,960; 5,741,554; and 5,738,728; which are fully incorporated by reference herein.

Microprojection coating formulations or solutions can also be applied using ink jet technology using known solenoid valve dispensers, optional fluid motive means and positioning means which is generally controlled by use of an electric field. Other liquid dispensing technology from the printing industry or similar liquid dispensing technology known in the art can be used for applying the pattern coating of this invention.

According to the invention, the coating formulation applied the microprojections 62, 64, 72, 74 to from the solid coating can similarly comprise any of the aforementioned coating formulations. The active agent can similarly comprise any of the aforementioned agents.

Referring now to FIG. 10A, there is shown the first step in the formation of yet another embodiment of the present invention. Sheet 90 is initially etched, punched or subject to laser drilling to form one or more groupings 94 of small openings 92. According to the invention, the openings can comprise various sizes and shapes.

The second step comprises the deformation or stretching of regions of sheet 90 proximate the groupings 94 to form one or more microprojections 96. A coating formulation is then preferably placed into the interior of one or more of microprojections 96. The formulation is dried to form a solid coating along the interior surface of one or more of microprojections 96.

As will be recognized by one having ordinary skill in the art, when the coated microprojections 96 are inserted into tissue, the coating is protected and not exposed to physical contact with the surrounding tissue; the openings 92 in microprojection 96 allowing for the subsequent dissolution of the coating by the interstitial fluid.

In additional envisioned embodiments of the invention, the coating formulation can also be applied to the outer surface of the microprojections 96.

Although the groupings 94 are shown in FIG. 10A comprise a circular arrangement of openings 92, the openings 92 and arrangements thereof can comprise various sizes and configurations. Clearly, the circular shape is most efficient, since it enables all of the openings 92 to be incorporated into the microprojection 96.

Though not shown, the area of sheet 90 that is deformed to create each microprojection 96 could be larger in area than any specific grouping 94. This would result in openings 92 only being disposed near the tip of microprojection 96.

Preferably, the microprojection 96 has a length less than approximately 1000 microns, more preferably, less than approximately 500 microns and a maximum diameter less than 200 microns, more preferably, less than 100 microns.

Though the general design of the invention disclosed herein is directed to a microprojection design that protects a coating containing an agent to be delivered, the invention can also be employed in conjunction with sampling a body fluid, such as interstitial fluid. The agent contained in the coating could be one that enhances production of a desired material, such as pilocarpine to enhance the production of sweat for cystic fibrosis testing, and/or one of the aforementioned an anticoagulant or anti-healing agents.

As will be appreciated by one having ordinary skill in the art, the microprojections of the present invention can be employed with passive transdermal devices and systems, such as the passive transdermal systems disclosed in Pat. Nos. 6,050,988, 6,083,196, 6,230,051 and 6,219,574, and active transdermal systems, such as the systems disclosed in Pat. Nos. 5,147,296, 5,080,646, 5,169,382 and 5,169,383; the disclosures of which are expressly incorporated herein in their entirety.

Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

1. A microprojection member for insertion into a biological surface, comprising:
a substantially conical shaped microprojection having a wall defining an interior region, said microprojection wall including a plurality of openings; and
a biocompatible coating disposed on said interior region of said microprojection.

2. The microprojection member of claim 1, wherein said microprojection is formed by deforming a region of a substantially planar sheet.

3. The microprojection member of claim 2, wherein said microprojection is constructed out of a material selected from the group consisting of stainless steel, titanium, nickel titanium alloys and like biocompatible materials.

4. The microprojection member of claim 1, wherein said microprojection is constructed out of a non-conductive material.

5. The microprojection member of claim 1, wherein said microprojection has a length less than approximately 1000 microns.

6. The microprojection member of claim 1, wherein said biocompatible coating is produced by applying a coating formulation on said microprojection.

7. The microprojection member of claim 6, wherein said coating formulation includes at least one biologically active agent selected from the group consisting of a hormone releasing hormone (LHRH), LHRH analog, vasopressin, desmopressin, corticotropin (ACTH), an ACTH analog, calcitonin, vasopressin, deamino [Val4, D-Arg8] arginine vasopressin, interferon alpha, interferon beta, interferon gamma, erythropoietin (EPO), granulocyte macrophage colony stimulating factor (GM-CSF), granulocyte colony stimulating factor (G-CSF), interleukin-10 (IL-10), glucagon, growth hormone releasing factor (GHRF), insulin, insulinotropin, calcitonin, octreotide, endorphin, TRN, NT-36 (chemical name: N-[[(s)-4-oxo-2-azetidinyl]carbonyl]-L-histidyl-L-prolinamide), liprecin, aANF, bMSH, somatostatin, bradykinin, somatotropin, platelet-derived growth factor releasing factor, chymopapain, cholecystokinin, chorionic gonadotropin, epoprostenol, hirulog, interferon, interleukin, menotropins, oxytocin, streptokinase, tissue plasminogen activator, urokinase, ANP, ANP clearance inhibitor, angiotensin II antagonist, antidiuretic hormone agonist, bradykinn antagonist, ceredase, CSI, calcitonin gene related peptide (CGRP), enkephalins, FAB fragment, IgE peptide suppressor, IGF-1, neurotrophic factor, colony stimulating factor, parathyroid hormone and agonist, parathyroid hormone antagonist, prostaglandin antagonist, pentigetide, protein C, protein S, renin inhibitor, thymosin alpha-1, thrombolytic, TNF, vasopressin antagonist analog, alpha-1 antitrypsin (recombinant), TGF-beta, fondaparinux, ardeparin, dalteparin, defibrotide, enoxaparin, hirudin, nadroparin, reviparin, tinzaparin, pentosan polysulfate, oligonucleotide and oligonucleotide derivatives, alendronic acid, clodronic acid, etidronic acid, ibandronic acid, incadronic acid, pamidronic acid, risedronic acid, tiludronic acid, zoledronic acid, argatroban, RWJ 445167, and RWJ-671818.

8. The microprojection member of claim 6, wherein said coating formulation includes at least one vaccine selected from the group consisting of flu vaccine, Lyme disease vaccine, rabies vaccine, measles vaccine, mumps vaccine, chicken pox vaccine, small pox vaccine, hepatitis vaccine, pertussis vaccine, diphtheria vaccine, recombinant protein vaccine, DNA vaccine and therapeutic cancer vaccine.

9. The microprojection member of claim 6, wherein said coating formulation includes at least one buffer selected from the group consisting of ascorbic acid, citric acid, succinic acid, glycolic acid, gluconic acid, glucuronic acid, lactic acid, malic acid, pyruvic acid, tartaric acid, tartronic acid, fumaric acid, maleic acid, phosphoric acid, tricarballylic acid, malonic acid, adipic acid, citraconic acid, glutaratic acid, itaconic acid, mesaconic acid, citramalic acid, dimethylolpropionic acid, tiglic acid, glyceric acid, methacrylic acid, isocrotonic acid, crotonic acid, angelic acid, hydracrylic acid, aspartic acid, glutamic acid, glycine and mixtures thereof.

10. The microprojection member of claim 6, wherein said coating formulation includes at least one surfactant selected from the group consisting of sodium lauroamphoacetate, sodium dodecyl sulfate (SDS), cetylpyridinium chloride (CPC), dodecyltrimethyl ammonium chloride (TMAC), benzalkonium, chloride, polysorbates and other sorbitan derivatives.

11. The microprojection member of claim 6, wherein said coating formulation includes at least one polymeric material selected from the group consisting of hydroxyethylcellulose (HEC), hydroxypropylmethylcellulose (HPMC), hydroxypropycellulose (HPC), methylcellulose (MC), hydroxyethylmethylcellulose (HEMC) and ethylhydroxy-ethylcellulose (EHEC).

12. The microprojection member of claim 6, wherein said coating formulation includes at least one hydrophilic polymer selected from the group consisting of hyroxyethyl starch, dextran, poly(vinyl alcohol), poly(ethylene oxide), poly(2-hydroxyethyl-methacrylate), poly(n-vinyl pyrolidone), polyethylene glycol and mixtures thereof.

13. The microprojection member of claim 6, wherein said coating formulation includes at least one biocompatible carrier selected from the group consisting of human albumin, bioengineered human albumin, polyglutamic acid, polyaspartic acid, polyhistidine, pentosan polysulfate, polyamino acids, sucrose, trehalose, melezitose, raffinose and stachyose.

14. The microprojection member of claim 6, wherein said coating formulation includes at least one stabilizing agent selected from the group consisting of a reducing sugar, non-reducing sugar and polysaccharide.

15. The microprojection member of claim 14, wherein said non-reducing sugar is selected from the group consisting of sucrose, trehalose, stachyose and raffinose.

16. The microprojection member of claim 14, wherein said polysaccharide is selected from the group consisting of dextran, soluble starch, dextrin and insulin.

17. The microprojection member of claim 14, wherein said reducing sugar is selected from the group consisting of apiose, arabinose, lyxose, ribose, xylose, digitoxose, fucose, quercitol, quinovose, rhamnose, allose, altrose, fructose, galactose, glucose, gulose, hamamelose, idose, mannose, tagatose, primeverose, vicianose, rutinose, scillabiose, cellobiose, gentiobiose, lactose, lactulose, maltose, melibiose, sophorose and turanose.

18. The microprojection member of claim 6, wherein said coating formulation includes at least one vasoconstrictor selected from the group consisting of amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and the mixtures thereof.

19. The microprojection member of claim 6, wherein said coating formulation includes at least one pathway patentency modulator selected from the group consisting of an osmotic agent, zwitterionic compound and anti-inflammatory agent.

20. The microprojection member of claim 19, wherein said anti-inflammatory agent is selected from the group consisting of betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate and prednisolone 21-succinate sodium salt.

21. The microprojection member of claim 19, wherein said pathway patentency modulator comprises an anticoagulant selected from the group consisting of citric acid, citrate salts, dextrin sulfate sodium, aspirin and EDTA.

22. The microprojection member of claim 6, wherein said coating formulation includes at least one solubilising/complexing agent selected from the group consisting of Alpha-Cyclodextrin, Beta-Cyclodextrin, Gamma-Cyclodextrin, glucosyl-alpha-Cyclodextrin, maltosyl-alpha-Cyclodextrin, glucosyl-beta-Cyclodextrin, maltosyl-beta-Cyclodextrin, hydroxypropyl beta-cyclodextrin, 2-hydroxypropyl-beta-Cyclodextrin, 2-hydroxypropyl-gamma-Cyclodextrin, hydroxyethyl-beta-Cyclodextrin, methyl-beta-Cyclodextrin, sulfobutylether-alpha-cyclodextrin, sulfobutylether-beta-cyclodextrin, and sulfobutylether-gamma-cyclodextrin. Most preferred solubilising/complexing agents are beta-cyclodextrin, hydroxypropyl beta-cyclodextrin, 2-hydroxypropyl-beta-Cyclodextrin and sulfobutylether7 beta-cyclodextrin.

23. The microprojection member of claim 6, wherein said coating formulation has a viscosity less than approximately 500 centipoise and greater than 3 centipose.

24. The microprojection member of claim 1, wherein said coating has a thickness less than 100 microns.
